(19) United States
(12) Patent Application Publication
(10) Pub. No.: US 2004/0073178 A1
(43) Pub. Date: Apr. 15, 2004

(54) Nasal irrigation fitting

(76) Inventors: Craig W. Anderson, Sandy, UT (US); Chad M. Adams, West Jordan, UT (US); Ralph L. Sonderegger, Farmington, UT (US)

Correspondence Address: GRANT R CLAYTON, CLAYTON HOWARTH & CANNON, PC, P O BOX 1909, SANDY, UT, 84091-1909, US

(21) Appl. No.: 10/440,463

(22) Filed: May 16, 2003

Related U.S. Application Data
(60) Provisional application No. 60/381,526, filed on May 16, 2002.

(51) Int. Cl. A61M 31/00

(52) U.S. Cl. 604/275

(57) ABSTRACT

A fitting is disclosed that adapts the wand of an oral irrigation device for use in nasal irrigation. An oral irrigation wand is inserted into a fitting, which is then placed in the nostril. The fitting is compatible with the wands of many brands and models of oral irrigation devices. The fitting dampens the change in amplitude of the pulsations from pulsating-type irrigation devices, resulting in a safer, more pleasant nasal irrigation experience. The fitting is removable and protects the user's nostril from contact with the wand and allows the wand to be used for both nasal and oral irrigation. The fitting allows for comfortable insertion into a nostril, providing a complete seal with both the nose and the nasal irrigation device, to promote both a tidy and effective nasal irrigation experiences. The fitting is sturdy, simple and easy to manufacture, providing for long life and performing well even with long, repetitive use.

### CROSS-REFERENCE TO RELATED APPLICATIONS

This application claims the benefit of U.S. Provisional Application No. 60/381,526, filed May 16, 2002, which is hereby incorporated by reference herein in its entirety, including but not limited to those portions that specifically appear hereinafter.

### BACKGROUND OF THE INVENTION

1. Field

This invention relates to a fitting suitable for adapting an oral irrigation device for use in nasal irrigation procedures. The present invention is more particularly, but not necessarily entirely, directed to a fitting that can couple with many models and types of oral irrigation wands and can reduce the velocity and dampen the pulsation of fluid emanating from an oral irrigation device while protecting the oral irrigation wand from contamination by nasal surfaces or secretions.

2. Background Art

Performing nasal irrigation procedures by humans has many desirable benefits. Nasal irrigation can help clear the nasal passages, thus promoting normal breathing and increasing the overall quality of life. Nasal irrigation can reduce nasal irritants that may contribute to allergic responses. Nasal irrigation is helpful in reducing the nasal load of micro-organisms that cause or contribute to infection. Nasal irrigation may also remove post-operative crusting and debris, which promotes successful healing after surgery.

Some sinus problems, such as sinusitis (inflammation of the sinuses), are exacerbated by bacterial accumulation in the nose and are often treated with antibiotics. Nasal irrigation can reduce or complement treatment with antibiotics by removing infected secretions. A recent medical study supports the benefits of nasal irrigation, reporting that 35% of subjects decreased use of sinus medications while using nasal irrigation. (Heatley, Diane G. et al. Otolaryngology: Head & Neck Surgery, vol 125, no. 1, pgs. 44-48, July 2001.)

Nasal irrigation is commonly performed by profusing a liquid through a patient's nose. During the procedure, the patient's head is positioned generally horizontally, face down. Liquid is pumped from an irrigation device, through an irrigation wand, into the nose where it bathes the sinus ostia, the nasopharynx, and flows thence outwardly through the other nostril, as is diagrammatically represented in FIG. 3. A light pressure of liquid is desirable, as too much force can be uncomfortable or harmful if one or both nostrils are clogged. The liquid flow desirably tends to clean and rinse mucous and debris from surfaces of the mucous membrane.

Many nasal irrigation devices, including bulb-type irrigators, manual spray mists, and powered irrigation devices exist on the market. However, many patients would prefer not to buy and store bulky or expensive equipment just for nasal irrigation. Oral irrigation devices, however, are extremely popular, and many patients already own an oral irrigation device. Oral irrigation devices are convenient to obtain as a number of manufacturers produce oral irrigation devices, each oral irrigation device being different than the others.

Although oral irrigation devices are convenient and readily available, adapting these devices for nasal irrigation presents problems which have been long felt in the art. Importantly, no oral irrigation device can be successfully used as a component of a nasal irrigation system without modification or supplementation.

Disadvantageously, each brand of oral irrigation device often offers several models to choose from, and in some cases each model or brand of oral irrigation device will change structure from time-to-time. A typical oral irrigation device includes multiple interchangeable wands (for example, providing one wand for each user) which are configured to direct the pulsating liquid stream into the desired location in the user's mouth. A structure for adapting an oral irrigation device for nasal irrigation would be most useful if it could be used with a variety of brands and models of oral irrigation devices.

A second disadvantage of adapting oral irrigation devices for use as a nasal irrigation system is that many oral irrigation devices emanate a pulsating flow of liquid, rather than a smooth flow of fluid. A typical oral irrigation device of this type pulses a liquid stream at a frequency rate ranging from 1300 to over 3000 pulses per minute and at a fairly intense amplitude. To a patient using the oral irrigation device as for nasal irrigation, this intense amplitude feels like staccato squirts of liquid into the nasal cavities which can be uncomfortable and possibly harmful for the patient. In comparison with oral irrigation, a less intense amplitude of pulsation is preferable for nasal irrigation, as the patient experiences a more smooth, flow-like fluid stream to irrigate the nostril.

Another disadvantage is that the oral irrigation wands require adaptation to be useful for nasal irrigation. The hard plastic tip of the wand is typically uncomfortable when inserted in the nose, and users generally prefer not to contaminate the oral irrigation wand with any purulent matter contained in the nostril. Also, the hard plastic irrigation tip of most oral irrigation wands do not provide a seal with the nostril, as is necessary to direct the flow upwards into the nasopharynx and out the other nostril (typically referred to as circular irrigation). The tip of the wand is typically curved, which requires the nasal irrigation user to hold the wand at an awkward angle to direct the stream of liquid into the nostril.

Another problem associated with adapting oral irrigation devices for nasal irrigation is that most oral irrigation devices emanate fluid at a velocity which may be uncomfortable or harmful for the patient during nasal irrigation. U.S. Pat. No. 5,899,878 (Glassman), (see FIG. 4) discloses an applicator for use with oral irrigation devices, that can interface with the nose and contains a structure for decreasing velocity.

A significant drawback of the Glassman applicator is that the mechanism that decreases the fluid velocity also decreases the volume flow rate, so the Glassman applicator does not provide sufficient volume for effectively irrigating the nasal passages. The Glassman applicator has both a nasal interface orifice where the liquid flows into the nostril and an exit port that is covered by the user's finger. The user can uncover the exit port to allow liquid to exit. This process is untidy as the liquid must drip into a sink or possibly onto the floor, but the main disadvantage is that the volume of liquid is reduced to the extent that volume is not sufficient to obtain the maximum benefit of nasal irrigation.

Furthermore, the applicator in Glassman is complicated, with many parts to manufacture and pieces that could potentially break off. Thus, the need exists for an apparatus that fits with a variety of existing oral irrigation wands that is simple to manufacture, reduces the change in pulsation amplitude, and reduces fluid flow volume rates without disadvantageously and unduly decreasing the volume of fluid flowing into the nose.

### SUMMARY OF THE INVENTION

The present invention provides a novel compatible fitting for effectively adapting oral irrigation devices for nasal irrigation. The novel design of the present invention meets the long-felt needs of nasal irrigation users: 1) one or more useful embodiments of the present invention is compatible with the different wands of a number of brands and models of oral irrigation devices; 2) one or more useful embodiments of the present invention decrease the velocity of the fluid while maintaining a substantially constant volume flow rate to provide a comfortable nasal irrigation experience; 3) at least one useful embodiment of the present invention reduces the change in pulsation amplitude of a pulsating oral irrigation device, such that a smoother stream of fluid flows into the nostril; 4) at least one useful embodiment of the present invention includes a removable fitting, which protects the nasal passageway from contact with an oral irrigation wand and allowing the oral irrigation wand to be utilized for both oral irrigation and nasal irrigation; 5) at least one useful embodiment of the present invention incorporates a fitting that is substantially vertically inserted into the nose and which provides a substantially complete seal with the oral irrigation wand, such that the user's arm is at a comfortable angle while performing nasal irrigation and the user does not have the annoyance of substantial amounts of liquid leaking from extra orifices; 6) at least one useful embodiment of the present invention of the present invention are sturdy, simple, and easy to manufacture, providing for long life and performing well even with repetitive and long use.

One illustrated embodiment of the present invention represented in the perspective view of FIG. 2, is a fitting into which an oral irrigation wand of an oral irrigation device is inserted. The fitting completely interfaces with the nostril, so that the oral irrigation tip orifice of the oral irrigation wand is protected from contamination by nasal secretions. The fitting generally comprises a hollow body shaped for comfortable insertion into either nostril. The fitting is desirably fabricated from a material that is effective and safe for use in medical devices.

Representative embodiments of the present invention include a conduit provided through a body of a fitting and a chamber provided in the body of the fitting in fluid communication with the conduit, such that liquid can flow from the chamber and into the conduit. It is useful for the chamber to be larger than the oral irrigation tip, so that the larger diameter of the chamber will modulate the pressure of the liquid flowing from the oral irrigation tip. The fitting has an oral irrigation wand orifice and a nasal interface orifice having a fluid connection there between, such that fluid may flow into an oral irrigation wand orifice and exit a nasal interface orifice, or fluid may flow into a nasal interface orifice and exit an oral irrigation wand orifice.

In use, the oral irrigation wand is inserted into the fitting. Fluid pulsates or flows from an oral irrigation device, into an oral irrigation wand, exits the oral irrigation tip orifice, flows into the chamber of the fitting, flows into the conduit of the fitting, and then exits the fitting from the nasal interface orifice to irrigate the nasal and exposed sinus passages. As the liquid pulsates or flows into the chamber and the conduit, both the fluid flow velocity and the intensity of the pulsations are dampened. This provides a much safer and more pleasant nasal irrigation experience for the patient than if the patient were to use the oral irrigation device unmodulated by the fitting.

The fitting generally comprises a structure that allows a substantially tight fit with a plurality of sizes and models of oral irrigation devices. It is preferred that the substantially tight fit allows less than 5 mL of fluid to escape per minute from any orifice other than the nasal interface orifice when the fitting is in use. This structure may illustratively comprises a cylindrical chamber, but may also be a spherical structure adapted for receiving a wand, an adjustable structure for receiving wands, or any other which can be arrived at by those skilled in the art that would allow the fitting to be compatible with a plurality of wands.

The chamber and conduit combination of the present invention greatly increases the durability and effectiveness of the structure. In previously available nasal irrigation fittings, the oral irrigation wand tip could disadvantageously pass directly through the body exposing the oral irrigation tip orifice to the inside the nostril. The fitting of the present invention overcomes such a problem by allowing the end of the oral irrigation tip to fit into the chamber, where it is completely concealed in the body. The resistence of the walls of the chamber prevent the tip from pushing through to emerge into the user's nasal passageway. Additionally, in the representative embodiments of the present invention, the oral irrigation tip is angled away from the nostril, so that it will not interface with the nostril even if the oral irrigation tip wears through the fitting.

Further features and advantages of the invention will be set forth in the description which follows, and in part will be apparent from the description, or may be learned by the practice of the invention without undue experimentation. The features and advantages of the invention may be realized and obtained by means of the instruments and combinations particularly pointed out in the appended claims.

### BRIEF DESCRIPTION OF THE DRAWINGS

The above and other features and advantages of the invention will become apparent from a consideration of the subsequent detailed description presented in connection with the accompanying drawings in which:

FIG. 1 is a side elevational view of a person using an embodiment of the present invention to adapt an oral irrigation device for nasal irrigation.

FIG. 2 is a perspective view of the user performing nasal irrigation with a nasal irrigation fitting of the present invention.

FIG. 3 is a diagrammatic representation of a user's head, showing in phantom image the approximate location of the sinuses and the flow of the liquid during nasal irrigation.

FIG. 4 depicts the prior art applicator for nasal irrigation disclosed in U.S. Pat. No. 5,899,878.

FIG. 5 is a top view of one embodiment of the nasal irrigation fitting.

FIG. 6 is a cross sectional view of one embodiment of the nasal irrigation fitting illustrated in FIG. 5 and taken along line 5-5.

FIG. 7 is a perspective view showing the swept curve design of the nasal irrigation fitting illustrated in FIG. 5.

FIGS. 8A-8E show oral irrigation wands for various brands of oral irrigation devices.

FIG. 9 is a cross sectional view of the nasal irrigation fitting illustrated in FIG. 5 with a first oral irrigation wand inserted therein.

FIG. 10 is a cross sectional view of the nasal irrigation fitting illustrated in FIG. 5 with a second oral irrigation wand inserted therein.

FIG. 11 is a perspective view of an oral irrigation wand inserted into a fitting of the present invention, with the fitting being inserted into a nostril.

FIG. 12 is a cross sectional view of the embodiment of the fitting shown in FIG. 5, showing representative dimensions.

### DETAILED DESCRIPTION OF THE DRAWINGS

For the purposes of promoting an understanding of the beneficial principles in accordance with the invention, reference will now be made to the embodiments illustrated in the drawings and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would normally occur to one skilled in the relevant art and having possession of this disclosure, are to be considered within the scope of the invention claimed.

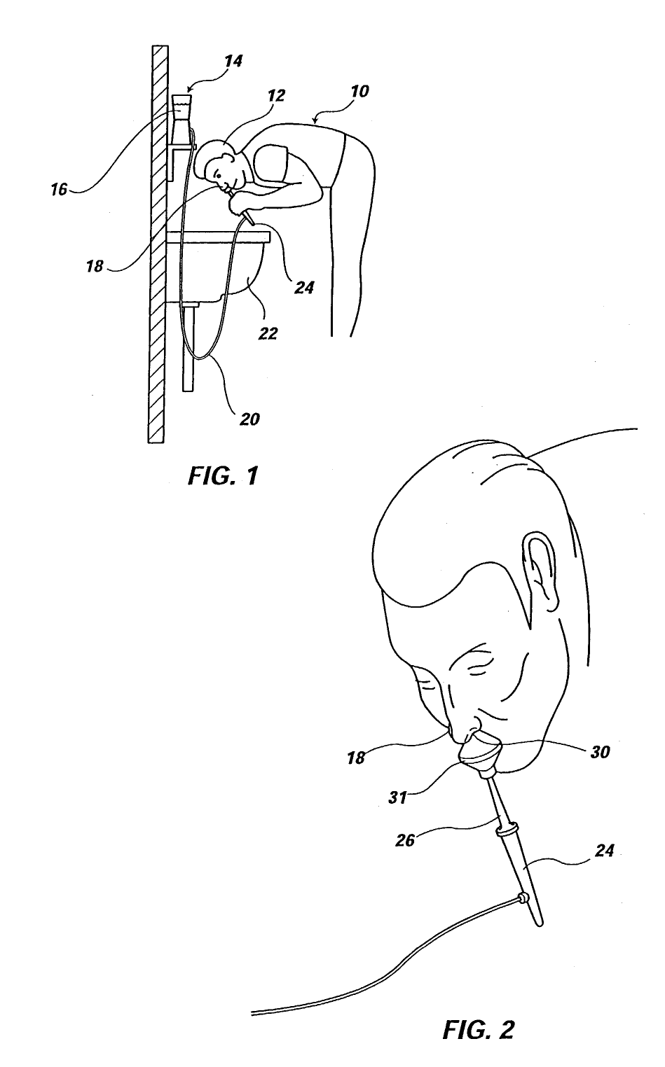

FIG. 1 shows a user 10 irrigating his nasal passages with the system of the present invention. The user 10 is oriented with his body bent in a downward position so that his head 12, and in particular his nose 18 is over a conventional basin 22. An oral irrigation device 14 feeds fluid from the fluid reservoir 16 to a flexible hose 20 to a connecting piece 24. Turning now to FIG. 2, the fluid flows from the connecting piece 24, through the oral irrigation wand 26, through the fitting 31 of the present invention, and into the nostril 30 of the nose 18.

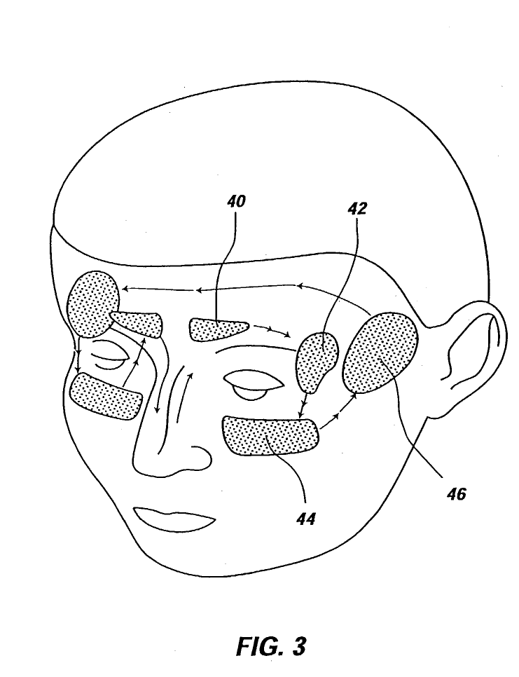

FIG. 3 diagrammatically illustrates the movement of the fluid as it moves through the sinuses, whose approximate location is shown in FIG. 3. Thus, after passing the nasolacrimal duct, the fluid typically passes, sequentially, the ostia of the frontal sinus indicated at 40, the ethnoids, indicated at 42, the maxillary indicated at 44 and the sphenoid indicated at 46. The fluid then moves past the outlet of the eustachian tube (not shown in FIG. 3) and thence through the nasopharynx to the upper posterior portion of the other nostril, and outwardly there through, passing the same ducts and ostia in inverse sequence, before being discharged outwardly of the second nostril. The pulsations present in the fluid flow can be effective to gently discharge crusted formations in the nasal passages, particularly near the sinus ostia, and the pulsations also stimulate the adjacent tissues.

FIG. 4 is a general representation of the applicator 50 described in the Glassman patent. The tube of the oral irrigation unit is represented at 20, the connecting piece of the irrigation unit is represented at 32. When an opening is completely covered by a slidable thumb rest 52, fluid moves through the applicator 50 and out the outlet port 56. When the opening under slidable thumb rest 52 is uncovered, fluid can exit the applicator 50 in a controlled manner to reduce the flow out of the outlet port 56.

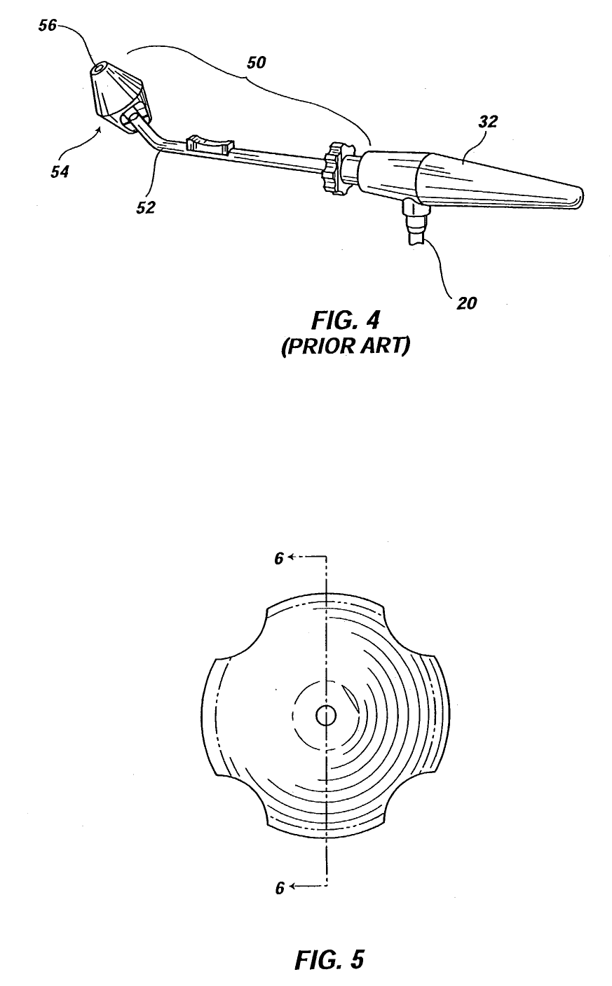

FIG. 5 is a top view of one embodiment of the nasal irrigation fitting. The cross sectional view of FIG. 6 is taken along line 6-6.

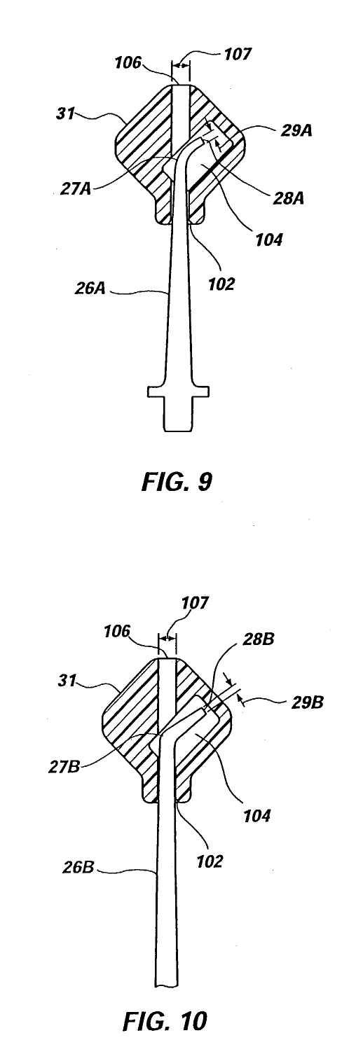

FIG. 6 shows a cross-sectional view of the embodiment of the present invention illustrated in FIG. 5 without an oral irrigation wand inserted. The oral irrigation wand can be readily inserted or removed as desired by the user. The oral irrigation wand orifice 102 creates a seal with an oral irrigation wand (see FIGS. 9-11) when the oral irrigation wand is inserted and provides a substantially tight fit. It is preferred that a substantially tight fit provide that less than 5 mL of liquid per minute can escape from any orifice other than the nasal interface orifice 106 when the oral irrigation wand is properly inserted into the fitting and the fitting is in use.

Although the oral irrigation wand is insertable and removable into the fitting shown in FIGS. 5-7, some users may choose to keep the oral irrigation wand inserted into the fitting. If the oral irrigation wand is never removed, the seal between the oral irrigation wand orifice 102 and an oral irrigation wand may tend to become less tight and fluid leakage occur. The present invention generally avoids this problem and those skilled in the art will readily recognize structures which can accommodate long term insertion of the oral irrigation wand, including the use of materials which prevent leakage and permanently affixing the oral irrigation wand to the fitting with adhesive, melting the material of the fitting to the wand, or other techniques that can be arrived at by those skilled in the art.

The chamber 104 represented in the cross sectional view of FIG. 6 is illustrative of a structure for operatively receiving an oral irrigation wand of an oral irrigation device. This structure is suitable in dimension to receive a plurality of oral irrigation wands from a variety of models and brands of oral irrigation devices. Although this structure is compatible with many wands, only one wand can be inserted into the structure at any one time. To insert a second wand, a user must remove the first wand and insert the second wand.

The chamber 104 represented in FIG. 6 comprises a first volume that is greater than zero. This volume may preferably comprise 4.663 in.³, but this volume may comprise any suitable volume such that the chamber may act as a reservoir which dampens the pulsation of a fluid emanating from an oral irrigation device.

It will be appreciated that the an oral irrigation device produces a fluid stream which pulsates and the flow of the fluid stream can be described as having a frequency, which can be described as the number of pulsations per minute, and an amplitude, which is the magnitude of each pulsation. Some oral irrigation devices can be set to provide a constant amplitude so that the fluid stream feels like a smooth stream to the user. If the amplitude of the fluid flow produced by the oral irrigation device changes greatly, then the nasal irrigation user can experience an unpleasant staccato sensation of liquid squirting into a nostril. The present invention avoids such disadvantages by including a structure, shown as a chamber 104 shown in the embodiment of FIG. 6, for dampening the pulsation of the fluid flow, such that the user experiences a smoother, more stream-like flow, rather than a staccato pulsation flow. It will be appreciated that the chamber is just one of many structures which perform the function of dampening the pulsation of the fluid emanating from the oral irrigation. It will also be appreciated that it is also within the scope of this invention to provide an embodiment of the present invention with an adjustable structure for decreasing pulsation, such as an engageable or disengageable flap, a valve, or some other structure which can be arrived at by those skilled in the art.

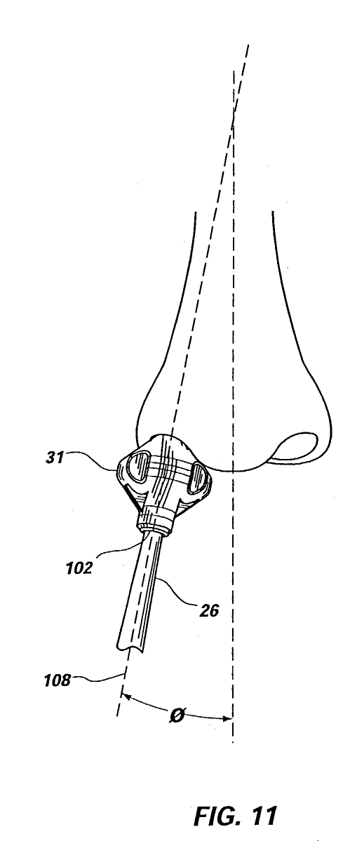

FIG. 6 illustrates a longitudinal axis 108 of the fitting, which will be discussed in greater detail in connection with FIG. 11. The fitting illustrated in FIG. 6 includes a nasal interface orifice 106, the nasal interface orifice 106 having a diameter indicated at 107.

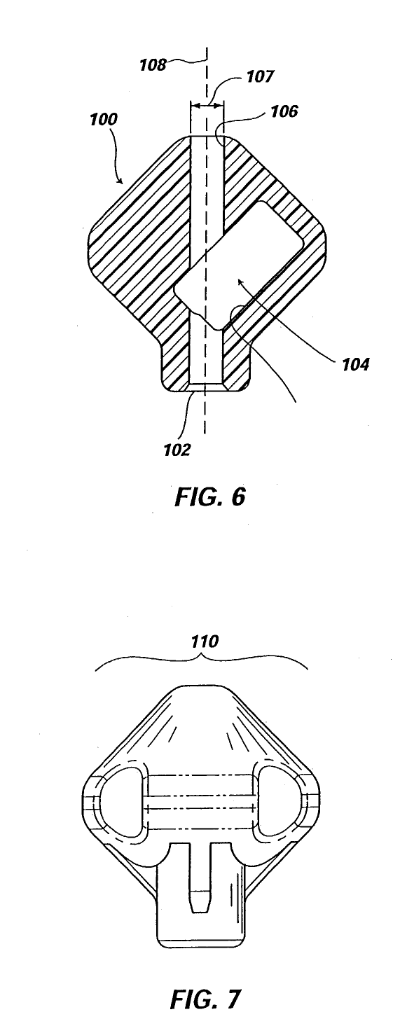

FIG. 7 is a side view of the fitting illustrated in FIGS. 5-6 showing the swept curve shape of the portion of the nasal irrigation fitting which interfaces with the nostril. The swept curve shape indicated by bracket 110 provides a substantial seal with any number of different nostrils found on different users and nostrils of different sizes. The substantial seal facilitates the flow of liquid upwards into the nasopharynx and out the other nostril for circular irrigation. The seal provided by the illustrated structure prevents substantial amounts of fluid from passing out of the nostril that is in contact with the nasal irrigation fitting. The swept curve shape 110 allows comfortable contact with the nostril, and does not allow the potentially damaging tip of the oral irrigation wand to touch the nostril. Although a swept curve shaped shown in FIG. 7 performs the necessary functions, it will be appreciated that many other shapes suitable for the comfort of the user and sealing with the nostril also fall within the scope of the present invention.

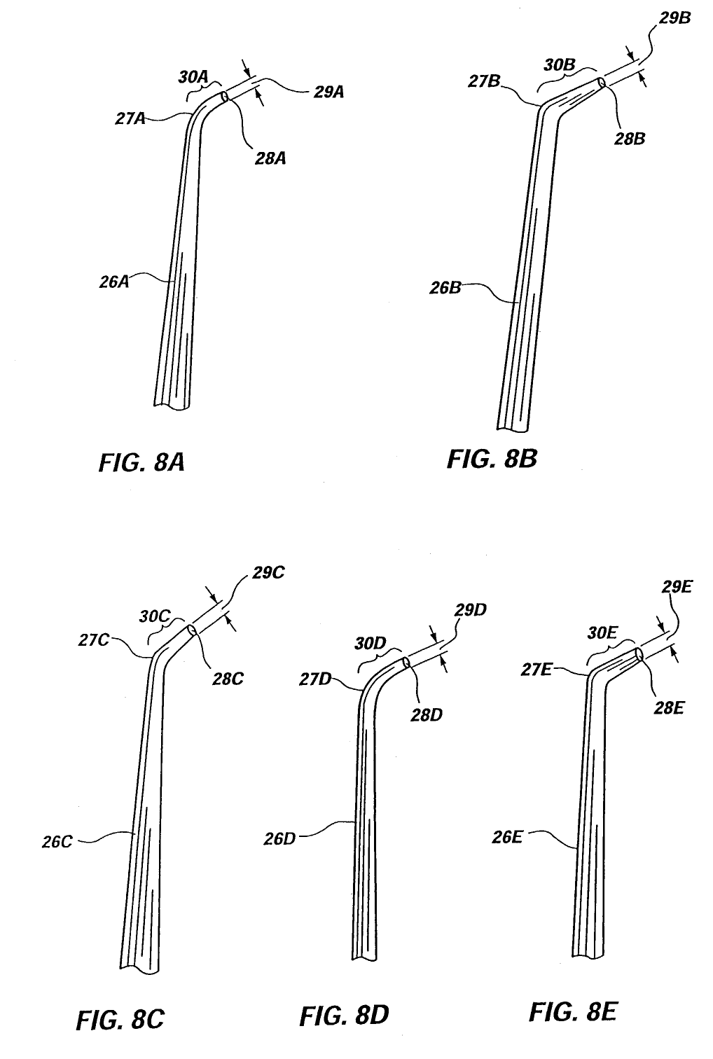

Turning now to FIGS. 8A-8E, a plurality of oral irrigation wands, each with their respective oral irrigation tip orifices and oral irrigation tip lengths, are shown. Each of a plurality of oral irrigation wands are shown generally at 26A-26E, respectively. Curvatures of the oral irrigation wands are represented at 27A-27E, respectively. The length of the oral irrigation tips of the plurality of oral irrigation wands is represented at 30A-30E, respectively. The oral irrigation tip orifices, through which the fluid exits, of the plurality of oral irrigation wands are represented at 28A-28E, respectively. The diameters of the oral irrigation tip orifices are represented at 29A-29E, respectively.

It will be appreciated that the dimensions of the wands 26A-26E are not equal. For example, the tip length 30A of the first oral irrigation wand 26A is not equal to the tip length 30B of the second oral irrigation wand. The diameter of the first oral irrigation tip orifice 29A is not equal to the diameter of the second oral irrigation tip orifice 29B. The first curvature 27A is not equal to the second curvature 27B. These wands 26A-E are merely illustrative of the plurality of wands with a plurality of different dimensions which are compatible with, and can be operatively received by, a single embodiment of the present invention.

FIGS. 9 and 10 illustrate the desirable compatibility of the embodiment of the present invention with different oral irrigation wands. FIG. 9 shows a first oral irrigation wand 26A inserted into the fitting 31. The first oral irrigation wand may illustratively comprise an INTERPLAK® oral irrigation wand. FIG. 10 shows a second oral irrigation wand 26B inserted into the fitting 31. The second oral irrigation wand may illustratively comprise a WATERPIK® oral irrigation wand.

Still referring to FIGS. 9 and 10, in order to ensure a comfortable nasal irrigation experience, it is useful for the diameter (29A-29E of FIGS. 8A-8E) of the oral irrigation tip orifice (28A-28E of FIGS. 8A-8E) to be greater than the diameter 107 (FIG. 9 and FIG. 10) of a nasal interface orifice (26A and 26B of FIG. 9 and FIG. 10), even though a plurality of diameters of oral irrigation tip orifices exist. If the volume of fluid flow of the fluid exiting the oral irrigation tip orifice is equal to a volume fluid flow exiting from a nasal interface orifice, and if the diameter of the nasal interface orifice is greater than the diameter of an oral irrigation tip orifice, then the velocity of a fluid exiting from the nasal interface orifice will be less than the velocity of a fluid exiting from the oral irrigation tip orifice.

The embodiment of the present invention provides advantages not heretofore available in the art. The illustrated embodiment provides that a high velocity of fluid provided by the oral irrigation device, such high velocity fluid often being harmful or uncomfortable for the nasal irrigation patient, is reduced to a safer, more pleasant velocity while maintaining the fluid volume necessary for effective nasal irrigation. However, it will be appreciated to those skilled in the art that many different alternative structures for decreasing fluid velocity while maintaining fluid flow through the fitting. It will also be appreciated by those skilled in the art that structures can be included in the embodiments of the present invention such that the amount which the velocity of the fluid flow is decreased can be adjusted by the user while maintaining a substantially constant volume. Thus, it is within the scope of the present invention to incorporate means for allowing the user to adjust the velocity of the fluid flow but substantially maintain the volume of the fluid flow.

Table A provides an exemplary range of measurements which may be used to fabricate the embodiment of the present invention utilizing the information provided herein with particular consideration for decreasing the velocity of the fluid exiting from the fitting while maintaining a desirable fluid flow volume. It is to be understood that all such measurements and dimensions provided herein are merely exemplary and are not intended to be limiting.

TABLE A

| | First | Second | Third |
|---|---|---|---|
| | Illustrative | Illustrative | Illustrative |
| | Measurement | Measurement | Measurement |
| Volume flow | .4 in³/sec.- | .04 in³/sec.- | .05 in³/sec. |
| rate | .6 in³/sec. | .06 in³/sec. | |
| Diameter of | .085 inches- | .09 inches- | .093 inches |
| the nasal | .098 inches | .095 inches | |
| interface | | | |
| orifice | | | |
| Diameter of | .02 inches- | .023 inches- | .024 inches |
| the oral | .03 inches | .025 inches | |
| irrigation | | | |
| tip orifice | | | |
| Velocity of | 30 in./sec.- | 6 in./second- | 7.361 in/sec. |
| fluid exiting | 125 in./sec. | 8 in./second | |
| the nasal | | | |
| interface | | | |
| orifice | | | |
| Velocity of | 1000 in./sec.- | 110 in/second- | 110.524 in./sec. |
| the fluid | 1250 in./sec. | 111 in./second | |
| exiting the | | | |
| oral | | | |
| irrigation | | | |
| tip orifice | | | |

FIG. 11 provides a perspective view of an oral irrigation wand and fitting ready to be used. The oral irrigation wand 26 is inserted into the fitting 31 in accordance with the present invention, the fitting 31 being provided with an oral irrigation wand orifice 102 and a nasal interface orifice 106. In FIG. 11, the user's nose is represented being divided at the midsagittal plane. The axis of a nostril lies along the midsagittal plane. As can be seen from FIG. 11, the axis of the fitting 108 is substantially parallel to the axis of the nostril, such that the angle θ is less than or equal to about 30 degrees with respect to the axis of the nostril. This allows for a substantial vertical insertion of the fitting into the nostril, so that the arm of the user can be held at a comfortable angle and the comfort of the nasal irrigation experience will be optimized for the user.

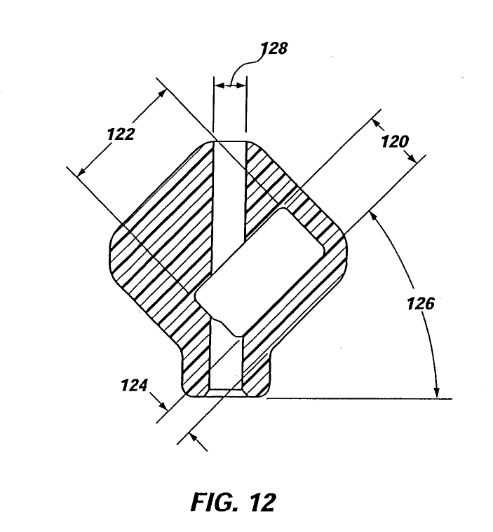

FIG. 12 is a cross sectional view of the embodiment of the present invention represented in FIG. 5. Provided in Table B are representative measurements for the of this embodiment. The measurements contained herein are intended only as exemplary measurements, and are not to be considered limiting of the structure which is considered to be within the scope of the present invention.

TABLE B

| | First | Second | Third |
|---|---|---|---|
| | Illustrative | Illustrative | Illustrative |
| | Measurement | Measurement | Measurement |
| Diameter of | .05-1.0 | .1-.4 inches | .2-.3 inches |
| the chamber | inches | | |
| 120 | | | |
| Length of the | .3-1.2 | .4-.7 inches | .5-.6 inches |
| chamber 122 | inches | | |
| Distance | .10-2.20 | .16-.18 | .17-.175 |
| between the | inches | inches | inches |
| fitting | | | |
| exterior and | | | |
| the chamber | | | |
| 124 | | | |
| Angle of the | 35 degrees- | 45 degrees- | 44 degrees- |
| chamber 126 | 65 degrees | 50 degrees | 46 degrees |
| Diameter of | .75 inches- | .08 inches- | .092 inches- |
| the nasal | 1.2 inches | 1.0 inches | .093 inches |
| interface | | | |
| orifice 128 | | | |

In accordance with the features and combinations described above, a useful method of nasal irrigation includes the steps of:

a) providing a source of nasal irrigation fluid;

b) inserting the oral irrigation wand into a fitting;

c) placing the fitting with an inserted oral irrigation wand into a nostril; and

d) pumping liquid with either a stream-like flow or a pulsating flow from the source of the nasal irrigation fluid though a fitting and into a nostril to irrigate the sinus cavities.

It will be appreciated that the above-recited steps, or the steps of any alternative method described herein, may be performed without regard to the order such steps are presented herein. Those skilled in the art will appreciate the most beneficial order in which the steps of the methods of the present invention can be carried out.

One illustrative method of making the fitting of the present invention will now be described. In this illustrative example, Santoprene® 8000 thermoplastic rubber, which is a thermoplastic elastomere of medical grade, is a useful material for making the fitting. However, other materials such as plastic, foam, or even metal can also be used within the scope of the present invention. It is generally preferred that the embodiments of the present invention be fabricated from a rubber material having a good hot tear strength for permitting rapid, easy removal of molded articles from injection and blow molding equipment and desirably provides for short molding cycle times. Another useful feature of the thermoplastic rubber material is a good sealing capacity (the ability to maintain a seal over a period of time). Sealing capacity is often measured by compression stress relaxation. Any number of materials are appropriate for use when fabricating embodiments of the present invention and in particular a material in which if the compression set is as low as possible, for example, a compression set between five and twenty at twenty-three degrees Celsius and twenty-two hours.

One illustrative method of making the fitting utilizing molding techniques is to use one metal pin to create the oral irrigation wand orifice the lower part of the conduit and the chamber and another metal pin to create the upper part of the conduit and the nasal interface orifice. After molding, several metal pins outside the fitting push the fitting off of the conduit-creating and chamber-creating pins. It is useful for the material from which the embodiments of the present invention are fabricated to have an ultimate elongation over 200% at twenty-three degrees Celsius, to allow the material sufficient stretch as to not be damaged as the fitting is pulled off the pins. One advantage of this illustrative method of manufacture is that the fitting can be made as a single piece, which is simple and inexpensive, yet creates a durable product. However, the fitting could also be fabricated from two or more pieces which are connected together using adhesive, parts are melted together, or using other methods known to those with skill in the art.

Those having ordinary skill in the relevant art will appreciate the advantages provided by the embodiments of the present invention. For example, it is an advantage of the present invention to provide a nasal irrigation fitting which is simple in design and manufacture. Another advantage of the present invention is providing a fitting which is compatible with the oral irrigation wands of a large number of oral irrigation devices. It is a further advantage of the present invention to provide a fitting that modulates the pulsation and pressure of the liquid which is pulsated from the oral irrigation device to provide a better nasal irrigation experience for the user. It is yet another advantage of the invention to provide a fitting that will protect the user's nostrils from contact with the oral irrigation wand, even when the oral irrigation wand is pushed into the fitting after repeated use.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been shown in the drawings and described above with particularity and detail, it will be apparent to those of ordinary skill in the art that numerous modifications, including, but not limited to, variations of size, materials, shape, form, function and manner of operation, assembly and use may be made without departing from the principles and concepts set forth herein.

What is claimed is:

1. A fitting for nasal irrigation operatively engaging with a plurality of oral irrigation wands, said plurality of oral irrigation wands comprising at least a first oral irrigation wand having a first diameter and a first length and a second oral irrigation wand having a second diameter and a second length, wherein the first diameter does not equal the second diameter and the first length does not equal the second length, the fitting comprising:
a body comprising an oral irrigation wand orifice and a nasal interface orifice, the oral irrigation wand orifice and the nasal interface orifice having a fluid connection therebetween;
a structure provided in said body to receive either the first oral irrigation wand or the second oral irrigation wand, such that the first and second wands are selectively and operatively received by the structure, regardless of the different configurations of the wands; and
such that when either the first oral irrigation wand is inserted into the oral irrigation wand orifice and is received by the structure or the second oral irrigation wand is inserted into the oral irrigation wand orifice and is received by the structure, any fluid flowing out of the first oral irrigation wand or the second oral irrigation wand passes through the body and out the nasal interface orifice, such that any one of the plurality of oral irrigation wands can operatively be received by the fitting and such that either the first oral irrigation wand or the second oral irrigation wand can be removed when desired.

2. A fitting as in claim 1, wherein said fitting comprises a swept shape for limited nostril penetration.

3. A fitting as in claim 1, wherein said fitting in use allows fluid to exit a nasal interface orifice, and wherein said fitting in use comprises a substantially tight fit such that no fluid can exit through the oral irrigation wand orifice.

4. A fitting as in claim 1, wherein said first oral irrigation wand comprises the dimensions of an INTERPLAK® oral irrigation wand.

5. A fitting as in claim 1, wherein said second oral irrigation wand comprises the dimensions of a WATERPIK® oral irrigation wand.

6. A fitting as in claim 1, wherein said fitting comprises a material with a percent compression set between 5 and 20 at twenty-three degrees C., and 22 hours.

7. A fitting as in claim 1, wherein said fitting comprises a material with a hot tear strength that permits the rapid, easy removal of molded articles from injection and blow molding equipment.

8. A fitting as in claim 1, wherein said fitting comprises a material with an ultimate elongation over 300% at twenty-three degrees Celsius.

9. A fitting as in claim 1, wherein said fitting comprises a material that can be inserted in the human nostril and a shape that can form a seal with a plurality of nostrils.

10. A fitting as in claim 1, wherein the fluid flowing out of one of the plurality of oral irrigation wands comprises a pulsating flow of fluid, and wherein the structure provided in the body comprises a chamber having a volume at least as great as a first volume, and wherein the first volume of the chamber is a reservoir which dampens the pulsation of the fluid emanating from an oral irrigation wand such that the amount of change in the amplitude of the pulsation of the fluid is decreased, while the frequency of the pulsation remains constant, such that the pulsation of the fluid exiting through a nasal interface orifice is less staccato than the pulsation of a fluid emanating from the oral irrigation wand.

11. A fitting as in claim 1, wherein said fitting is molded using injection molding with a material that permits removal of molded articles from injection and blow molding equipment, wherein the material of the fitting has an ultimate elongation of over 200% at twenty-three degrees Celsius.

12. A fitting as in claim 1, wherein said fitting is molded using one or more metal pins.

13. A fitting for nasal irrigation operatively engaging an oral irrigation wand connected to an oral irrigation device, the oral irrigation device providing a pulsating flow of fluid through the oral irrigation wand, the fitting comprising:
a body comprising an oral irrigation wand orifice, the oral irrigation wand being receivable therein, and a nasal interface orifice, the oral irrigation wand orifice and the nasal interface orifice having a fluid connection therebetween;
a chamber provided in the body, the chamber having a volume at least as great as a first volume; and
the oral irrigation device causes the fluid to pulsate through the oral irrigation wand at a first frequency, into the chamber and through the nasal interface orifice, and wherein the first volume of the chamber acts as a reservoir which dampens the pulsation of the fluid emanating from the oral irrigation wand such that the frequency of the pulsation is not greater than the first frequency and such that the amount of change of the amplitude of the pulsation of the fluid is decreased, such that the pulsation of the fluid exiting through the nasal interface orifice is less staccato than the pulsation of the fluid emanating from the oral irrigation wand.

14. A fitting as in claim 13, wherein said fitting comprises a swept shape for limited nostril penetration.

15. A fitting as in claim 13, wherein said fitting in use allows fluid to exit a nasal interface orifice, and wherein said fitting in use does not allow fluid to exit an oral irrigation wand orifice.

16. A fitting as in claim 13, wherein said fitting comprises a material with a percent compression set between 5 and 20 at twenty-three degrees Celsius, and 22 hours.

17. A fitting as in claim 13, wherein said fitting comprises a material with a hot tear strength that permits the rapid, easy removal of molded articles from injection and blow molding equipment.

18. A fitting as in claim 13, wherein said fitting comprises a material with an ultimate elongation over 300% at twenty-three degrees Celsius.

19. A fitting as in claim 13, wherein said fitting comprises a material that can be inserted in the human nostril and a shape that can form a seal with a plurality of nostrils.

20. A fitting as in claim 13, wherein at least a first oral irrigation wand can be inserted and removed from the chamber and a second oral irrigation wand can be inserted and removed from the chamber and a third oral irrigation wand can be inserted and removed from the chamber, and wherein a first length and a first diameter of the first oral irrigation wand is not equal to a second length and a second diameter of a second oral irrigation wand and wherein a third length and a third diameter of the third oral irrigation wand is not equal to the second length and the second diameter or the first length and the first diameter.

21. A fitting as in claim 13, wherein the fitting can be inserted into a nostril, and wherein the fitting comprises a first longitudinal axis, and a nostril comprises a second longitudinal axis created along the midsagittal plane, and wherein the first and second longitudinal axes are substantially parallel when an oral irrigation wand is inserted into the fitting and when the fitting is inserted into the nostril.

22. A fitting as in claim 13, wherein the oral irrigation wand comprises an oral irrigation tip orifice with a first diameter and a nasal interface orifice comprises a second diameter, and wherein the second diameter is greater than the first diameter.

23. A fitting as in claim 22, wherein the first diameter is between 0.02 inches and 0.03 inches and the second diameter is between 0.09 inches and 0.1 inches.

24. A fitting as in claim 13, wherein said fitting is molded using injection molding with a material that permits removal of molded articles from injection and blow molding equipment, wherein the material of the fitting has an ultimate elongation of over 200% at twenty-three degrees Celsius.

25. A fitting as in claim 13, wherein said fitting is molded using one or more metal pins.

26. A method of performing nasal irrigation, utilizing an oral irrigation wand and a fitting, the method comprising the steps of:
a) Inserting the oral irrigation wand having a first oral irrigation tip orifice with a first diameter into the fitting, said fitting comprising an oral irrigation wand orifice and a nasal interface orifice, the oral irrigation wand orifice and the nasal interface orifice having a fluid connection therebetween, wherein said nasal interface orifice comprises a second diameter;
b) Placing the fitting in a nostril having a longitudinal axis along a midsagittal plane, such that a first longitudinal axis of the fitting is substantially parallel to a second longitudinal axis of the nostril;
c) Engaging the oral irrigation device such that fluid flows from the oral irrigation device, into the oral irrigation wand, through the oral irrigation tip orifice having a first diameter, into the fitting and then out the nasal interface orifice, wherein the second diameter of the nasal interface orifice is greater than the first diameter of the oral irrigation tip orifice; and
d) Irrigating the nostril with the fluid emanating from the nasal interface orifice.

27. A method as in claim 26, further comprising the steps of:
e) Removing the oral irrigation wand from the fitting.

28. A method as in claim 26, wherein the fitting comprises a swept shape for limited nostril penetration.

29. A method as in claim 26, wherein the fitting allows fluid to exit a nasal interface orifice, and wherein said fitting in use does not allow fluid to exit an oral irrigation wand orifice.

30. The method as in claim 26, wherein the fitting comprises a material with a percent compression set between 5 and 20 at twenty-three degrees Celsius, and 22 hours.

31. The method as in claim 26, wherein the fitting comprises a material with a hot tear strength that permits the rapid, easy removal of molded articles from injection and blow molding equipment.

32. A method as in claim 26, wherein the fitting comprises a material with an ultimate elongation over 300% at twenty-three degrees Celsius.

33. A method as in claim 26, wherein the fitting comprises a material that can be inserted in the human nostril and a shape that can form a seal with a plurality of nostrils.

34. A method as in claim 26, wherein a fluid flowing out of the oral irrigation wand comprises a pulsating flow of fluid, and wherein the fitting comprises a chamber having a volume at least as great as a first volume, and wherein the first volume of the chamber is a reservoir which dampens the pulsation of the fluid emanating from an oral irrigation wand such that the amount of change of the amplitude of the pulsation of the fluid is decreased, while the frequency of the pulsation remains constant, such that the pulsation of the fluid exiting through a nasal interface orifice is less staccato than the pulsation of a fluid emanating from the oral irrigation wand.

35. A method as in claim 26, wherein said fitting is molded using injection molding with a material that permits removal of molded articles from injection and blow molding equipment, wherein the material of the fitting has an ultimate elongation of over 200% at twenty-three degrees Celsius.

36. A method as in claim 26, wherein said fitting is molded using one or more metal pins.

37. A method as in claim 26, wherein the first longitudinal axis is within 20 degrees of the second longitudinal axis of the nostril, with respect to the midsagittal plane.

38. A method as in claim 26, wherein step (a) is completed prior to step (b).

39. A method as in claim 26, wherein step (b) is completed prior to step (a).

40. A method of performing nasal irrigation using a fitting that operatively engages a plurality of oral irrigation wands, said plurality of oral irrigation wands comprising at least a first oral irrigation wand having a first length and a first oral irrigation tip orifice, wherein the first oral irrigation tip orifice comprises a first diameter, said plurality of oral irrigation wands further comprising a second oral irrigation wand having a second length and a second oral irrigation tip orifice, having a second diameter, wherein the first diameter does not equal the second diameter and the first length does not equal the second length, the method comprising the steps of:
a) Inserting the first oral irrigation wand into a fitting, said fitting comprising an oral irrigation wand orifice and a nasal interface orifice, the oral irrigation wand orifice and the nasal interface orifice having a fluid connection therebetween, wherein said nasal interface orifice comprises a third diameter; and wherein said third diameter is greater than the first diameter and wherein said third diameter is greater than the second diameter;
b) Placing the fitting in a nostril; and
c) Engaging the oral irrigation device into which either a first oral irrigation wand or a second oral irrigation wand is selectively inserted, such that fluid either flows from the oral irrigation device, and out the first oral irrigation tip orifice when the first oral irrigation wand is inserted into the fitting, or such that the fluid flows from the oral irrigation device, out the second oral irrigation tip orifice when the second oral irrigation wand is inserted into the fitting; wherein the fluid then flows into the fitting, and out through the nasal interface orifice to irrigate the nostril.

41. A method as in claim 40, further comprising the steps of:
e) Removing the oral irrigation wand from the fitting.

42. A method as in claim 40, wherein the fitting comprises a swept shape for limited nostril penetration.

43. A method as in claim 40, wherein the fitting allows fluid to exit a nasal interface orifice, and wherein said fitting in use does not allow fluid to exit an oral irrigation wand orifice.

44. The method as in claim 40, wherein the fitting comprises a material with a percent compression set between 5 and 20 at twenty-three degrees Celsius, and 22 hours.

45. The method as in claim 40, wherein the fitting comprises a material with a hot tear strength that permits the rapid, easy removal of molded articles from injection and blow molding equipment.

46. A method as in claim 40, wherein the fitting comprises a material with an ultimate elongation over 300% at twenty-three degrees Celsius.

47. A method as in claim 40, wherein the fitting comprises a material that can be inserted in the human nostril and a shape that can form a seal with a plurality of nostrils.

48. A method as in claim 40, wherein a fluid flowing out one of the plurality of oral irrigation wand orifices comprises a pulsating flow of fluid, and wherein the fitting comprises a chamber having a volume at least as great as a first volume, and wherein the first volume of the chamber is a reservoir which dampens the pulsation of the fluid emanating from an oral irrigation wand such that the amplitude of the pulsation of the fluid is decreased, while the frequency of the pulsation remains constant, such that the pulsation of the fluid exiting through a nasal interface orifice is less staccato than the pulsation of a fluid emanating from the oral irrigation wand.

49. A method as in claim 40, wherein said fitting is molded using injection molding with a material that permits removal of molded articles from injection and blow molding equipment, wherein the material of the fitting has an ultimate elongation of over 200% at twenty-three degrees Celsius.

50. A method as in claim 40, wherein the fitting is molded using one or more metal pins.

51. A method as in claim 40, wherein step (a) is completed prior to step (b).

52. A method as in claim 40, wherein step (b) is completed prior to step (a).

53. A method of performing nasal irrigation, utilizing an oral irrigation wand and a fitting, having a conduit and a chamber in communication with the conduit, the method comprising the steps of:
a) Preventing an oral irrigation tip orifice of an oral irrigation wand of an oral irrigation device from touching a nostril or nasal secretions by inserting the oral irrigation wand into the chamber of the fitting, wherein the fitting comprises a nasal interface orifice in communication with the conduit;
b) Inserting the fitting into a nostril;
c) Engaging an oral irrigation device such that fluid flows from the oral irrigation device and through the oral irrigation wand, out the oral irrigation tip orifice, into the chamber, into the conduit and then out through the nasal interface orifice to irrigate the nostril; and
d) Removing the oral irrigation wand from the fitting.

54. The method of claim 53, the steps further comprising:
e) Using the oral irrigation wand to perform oral irrigation.

55. The method of claim 53, wherein the nostril comprises a first longitudinal axis along a midsagittal plane and the fitting comprises a second longitudinal axis, and wherein the first longitudinal axis and the second longitudinal axis are substantially parallel when an oral irrigation wand is inserted into the chamber of the fitting and when the fitting is inserted in the nostril.

56. A method as in claim 53, wherein a fluid flowing out of the oral irrigation tip orifice comprises a pulsating flow of fluid, and wherein the fitting comprises a chamber having a volume at least as great as a first volume, and wherein the first volume of the chamber is a reservoir which dampens the pulsation of the fluid emanating from the oral irrigation wand orifice such that the amount of change of the amplitude of the pulsation of the fluid is decreased, while the frequency of the pulsation remains constant, such that the pulsation of the fluid exiting through a nasal interface orifice is less staccato than the pulsation of a fluid emanating from the oral irrigation wand orifice.

57. A fitting for performing nasal irrigation using an oral irrigation wand having an oral irrigation tip orifice comprising:
a fitting having an oral irrigation wand orifice and a nasal interface orifice;
said fitting further comprising a first longitudinal axis and a conduit;
wherein the fitting comprises a swept shape suitable for insertion into a nostril, the nostril having a second longitudinal axis along a midsagittal plane, wherein the first longitudinal axis is substantially parallel to the second longitudinal axis when the fitting is inserted into the nostril; and
a chamber in communication with the conduit that prevents the oral irrigation tip orifice from touching a nostril surface or matter contained in the nostril when the oral irrigation wand is inserted in the fitting.

58. A fitting as in claim 57, wherein the first longitudinal axis is withing 30 degrees of the second longitudinal axis, with respect to the midsagittal plane.

59. A fitting as in claim 57, wherein the chamber comprises dimensions such that a plurality of oral irrigation wands can be inserted into the chamber.

60. A fitting as in claim 57, wherein the dimensions of the chamber are between 0.120 inches and 0.140 inches in diameter and 0.4 inches and 0.6 inches in length.

61. A nasal irrigation system for decreasing a velocity of a fluid emanating from an oral irrigation tip orifice of an oral irrigation wand of an oral irrigation device, such that the velocity is suitable for nasal irrigation, the system comprising:
a nasal irrigation system wherein a fluid flows from an oral irrigation device, through an oral irrigation wand, then out of a nasal interface orifice and into a nostril, wherein the oral irrigation wand has an oral irrigation tip orifice;
the fitting comprising an oral irrigation wand orifice and the nasal interface orifice, the oral irrigation wand orifice and the nasal interface orifice having a fluid connection therebetween;
wherein a first diameter of the nasal interface orifice is greater than a second diameter of an oral irrigation tip orifice; and
wherein a first velocity of a fluid exiting through the oral irrigation tip orifice is greater than a second velocity of a fluid exiting through a nasal interface orifice when a first volume flow rate of a fluid passing through the oral irrigation tip orifice is equal to a second volume flow rate of a fluid passing through the nasal interface orifice.

62. A nasal irrigation system as in claim 61, wherein the first diameter is between 0.085 inches and 0.125 inches and the second diameter is between 0.0126 inches and 0.04 inches.

63. A nasal irrigation system as in claim 61, wherein the first velocity is between 100 in/sec and 1200 in/sec and wherein a second velocity is between 6 in/sec and 75 in/sec.

64. A nasal irrigation system as in claim 61, wherein the first volume flow rate is between 0.04 in³/sec and 0.06 in³/sec., and wherein a first diameter is between 0.09 inches and 0.095 inches and a second diameter is 0.023 inches and 0.025 inches, and wherein a first velocity is between 110 in/sec and 111 in/sec and a second velocity is between 6 in/sec and 8 in/sec.

65. A nasal irrigation system as in claim 61, wherein the first volume flow rate is between 0.4 in³/sec. and 0.6 in³/sec, and wherein a first diameter is between 0.085 inches and 0.098 inches and a second diameter is 0.02 inches and 0.03 inches, and wherein a first velocity is between 1000 in/sec and 1250 in/sec and a second velocity is between 30 in/sec and 125 in/sec.

66. A nasal irrigation system for decreasing a velocity of a fluid emanating from an oral irrigation tip orifice of an oral irrigation wand of an oral irrigation device, such that the velocity is suitable for nasal irrigation, the system comprising:
a fluid stream flowing from an oral irrigation device, through an oral irrigation wand having an oral irrigation tip orifice, wherein the fluid emanating from the oral irrigation tip orifice comprises a first velocity;
a structure for receiving said fluid, the structure comprising a nasal interface orifice;
wherein a fluid stream flow through the nasal interface orifice has a second velocity, and wherein the second velocity is less than the first velocity;
wherein the structure for receiving the fluid comprises means for decreasing the first velocity, and wherein said means for decreasing the first velocity is not adjustable when the oral irrigation wand is inserted in the fitting; and
and wherein the structure is engageable and disengageable with a wand of an oral irrigation device, such that the oral irrigation wand can be inserted into the structure and removed from the structure.

67. A nasal irrigation system as in claim 66, wherein the first diameter is between 0.085 inches and 0.125 inches and the second diameter is between 0.0126 inches and 0.04 inches.

68. A nasal irrigation system as in claim 66, wherein the first velocity is between 100 in/sec and 1200 in/sec and wherein a second velocity is between 6 in/sec and 75 in/sec.

69. A nasal irrigation system as in claim 66, wherein the first volume flow rate is between 0.04 in³/sec and 0.06 in³/sec., and wherein a first diameter is between 0.09 inches and 0.095 inches and a second diameter is 0.023 inches and 0.025 inches, and wherein a first velocity is between 110 in/sec and 111 in/sec and a second velocity is between 6 in/sec and 8 in/sec.

70. A nasal irrigation system as in claim 69, wherein the first volume flow rate is between 0.3 in³/sec. and 0.7 in³/sec, and wherein a first diameter is between 0.075 inches and 0.2 inches and a second diameter is 0.015 inches and 0.045 inches, and wherein a first velocity is between 1000 in/sec and 1500 in/sec and a second velocity is between 10 in/sec and 900 in/sec.

71. A nasal irrigation system for decreasing a velocity of a fluid emanating from an oral irrigation tip orifice of an oral irrigation wand of an oral irrigation device, wherein the nasal irrigation system comprises a fitting, and wherein a plurality of oral irrigation wands can be inserted into the fitting, said nasal irrigation system comprising:
a fitting having a first longitudinal axis comprised of a material suitable for insertion into a nostril having a second longitudinal axis, wherein the first longitudinal axis and the second longitudinal axis are substantially parallel when the fitting is inserted into the nostril;
a chamber provided in the fitting, the chamber having a volume at least as great as a first volume such that a plurality of oral irrigation wands can be selectively inserted into the chamber;
a plurality of oral irrigation wands of one or more oral irrigation devices comprising a first oral irrigation wand with a first length and a first oral irrigation tip orifice with a first diameter and a second oral irrigation wand with a second length and a second oral irrigation tip orifice with a second diameter, wherein the first length and a first diameter are not equal to the second length and the second diameter;
the fitting having an oral irrigation wand orifice and a nasal interface orifice, wherein the oral irrigation orifice and the nasal interface orifice have a fluid connection therebetween;
the nasal interface orifice comprising a third diameter, wherein the third diameter is greater than the first diameter and the second diameter of the first oral irrigation wand and the second oral irrigation wand, respectively;
wherein the chamber prevents the first oral irrigation tip orifice from touching the nostril when the first oral irrigation wand is inserted into the chamber and wherein the chamber prevents the second oral irrigation tip orifice from touching the nostril when the second oral irrigation wand is inserted into the chamber; and
and wherein said nasal irrigation system is suitable for nasal irrigation when a fluid emanates from a nasal irrigation device, through one of a plurality of oral irrigation wands, through one of a plurality of oral irrigation tip orifices, through the chamber of the fitting, out the nasal interface orifice and into the nostril.
